SEQUENCE LISTING (4 sequences)

SEQ ID NO: 1 - DNA, 1164 bp, Homo sapiens
atgaatcggcaccatctgcaggatcactttctggaaatagacaagaagaactgctgtgtgttccgagatgacttcattgccaaggtgttgccgccggtgttggggctggagtttatctttgggcttctgggcaatggccttgccctgtggattttctgtttccacctcaagtcctggaaatccagccggattttcctgttcaacctggcagtagctgactttctactgatcatctgcctgccgttcgtgatggactactatgtgcggcgttcagactggaactttggggacatcccttgccggctggtgctcttcatgtttgccatgaaccgccagggcagcatcatcttcctcacggtggtggcggtagacaggtatttccgggtggtccatccccaccacgccctgaacaagatctccaattggacagcagccatcatctcttgccttctgtggggcatcactgttggcctaacagtccacctcctgaagaagaagttgctgatccagaatggccctgcaaatgtgtgcatcagcttcagcatctgccataccttccggtggcacgaagctatgttcctcctggagttcctcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctcaaataaccattccaagaagggacattgtcaccaagaaccagcatctctggagaaacagttgggctgttgcatcgagtaa

SEQ ID NO: 2 - DNA, 1092 bp, Homo sapiens
atgaatcggcaccatctgcaggatcactttctggaaatagacaagaagaactgctgcgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaa

SEQ ID NO: 3 - protein, 387 aa, Homo sapiens
MNRHHLQDHFLEIDKKNCCVFRDDFIAKVLPPVLGLEFIFGLLGNGLALWIFCFHLKSWKSSRIFLFNLAVADFLLIICLPFVMDYYVRRSDWNFGDIPCRLVLFMFAMNRQGSIIFLTVVAVDRYFRVVHPHHALNKISNWTAAIISCLLWGITVGLTVHLLKKKLLIQNGPANVCISFSICHTFRWHEAMFLLEFLLPLGIILFCSARIIWSLRQRQMDRHAKIKRAITFIMVVAIVFVICFLPSVVVRIRIFWLLHTSGTQNCEVYRSVDLAFFITLSFTYMNSMLDPVVYYFSSPSFPNFFSTLINRCLQRKMTGEPDNNRSTSVELTGDPNKTRGAPEALMANSGEPWSPSYLGPTSNNHSKKGHCHQEPASLEKQLGCCIE

SEQ ID NO: 4 - protein, 363 aa, Homo sapiens [sequence omitted]

(19) United States
(12) Patent Application Publication
(10) Pub. No.: US 2008/0089881 A1
(43) Pub. Date: Apr. 17, 2008

(54) Treatment and Diagnostics of Inflammatory Diseases

(76) Inventors: Jun Li, Danbury, CT (US); Xiang John Li, Danbury, CT (US); Baerbel Losacco, New Milford, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(21) Appl. No.: 11/567,341

(22) Filed: Dec. 6, 2006

Related U.S. Application Data
(60) Provisional application No. 60/748,313, filed on Dec. 7, 2005.

(51) Int. Cl. A61K 31/70; A61K 38/00; A61K 39/395; A61P 29/00; C12Q 1/68

(52) U.S. Cl. 424/130.1; 435/6; 514/12; 514/2; 514/44

(57) ABSTRACT

A method of determining whether a subject is suffering from or at risk for developing inflammatory diseases by determining the level of HM74 and/or HM74A in tissue damage. Also disclosed are a method of identifying a compound for treating inflammatory diseases, a method of treating inflammatory diseases, and a pharmaceutical composition or a packaged product for treating inflammatory diseases.

### RELATED APPLICATIONS

This application claims benefit from U.S. Provisional Application No. 60/748,313, filed on Dec. 7, 2005.

### BACKGROUND

HM74 was first cloned as an orphan GPCR from a human monocyte cDNA library in 1993 (Nomura H et al, 1993, Int Immunol. 5:1239-1249). Human HM74 has two isoforms, named as HM74 and HM74A. HM74 is a low affinity receptor for nicotinic acid while HM74A acts as a high affinity receptor for nicotinic acid (Wise A et al J Biol Chem. 2003 278:9869-9874). In mice lacking PUMA-G (the mouse analogue of human HM74A), the nicotinic acid-induced decrease in free fatty acid (FFA) and triglyceride plasma levels was abrogated, indicating that PUMA-G mediates the anti-lipolytic and lipid-lowering effects of nicotinic acid in vivo (Nat Med. 2003 9:352-355). The cDNA sequences between human HM74 (Seq ID #1) and human HM74A (Seq. ID #2) are very similar, with HM74A having 15 nucleotide changes and 5 bases insertion. HM74 and HM74A have been shown to have similar mRNA tissue distribution and chromosomal location (Wise A et al J Biol Chem. 2003 278:9869-9874). But the tissue selection in this study was limited to only normal tissues.

By gene expression studies, we have identified that HM74 mRNA is significantly induced in tissues from several inflammatory diseases such as psoriasis, Crohn's disease, ulcerative colitis and multiple sclerosis. Human HM74 has two isoforms, HM74A (high affinity to nicotinic acid) and HM74 (weak affinity to nicotinic acid). With TaqMan primers designed specifically against each isoform, we observed that both HM74 and HM74A are highly induced in inflamed Inflammatory Bowel Diseased (IBD) colons, and HM74A is highly induced in activated human Th2 cells.

### SUMMARY

This invention relates to use of GPCR genes, HM74 and/or HM74A as targets for treating inflammatory diseases.

In one aspect, the invention features a method of determining whether a subject is suffering from or at risk for developing inflammatory diseases such as psoriasis, Crohn's disease, ulcerative colitis, multiple sclerosis and irritable bowel disease. In one embodiment, the method includes providing a tissue sample from a subject and determining the gene expression level of HM74 and/or HM74A in the sample. If the gene expression level of HM74 and/or HM74A in the sample is higher than that in a sample from a normal subject, it indicates that the subject is suffering from or at risk for developing inflammatory diseases. The gene expression level of HM74 and/or HM74A can be determined by measuring the amount of the mRNA or the protein of HM74 (Seq. ID #3) and/or HM74A (SEQ. ID #4). The mRNA level can be determined by methods well known in the art such as by in situ hybridization, TaqMan real time RT-PCR, or northern blot analysis. The protein level can be determined, e.g., by western blot analysis. In another embodiment the method includes providing a sample from a subject and determining the protein activity level of HM74 and/or HM74A in the sample. If the protein activity level of HM74 and/or HM74A in the sample is higher than that in a sample from a normal subject, it indicates that the subject is suffering from or at risk for developing inflammatory diseases. The protein activity level of HM74 and/or HM74A can be determined, e.g., by measuring the binding of nicotinic acid, or by measuring GDP-GTP exchange on G-protein subunits following ligand-induced activation of HM74/HM74A,

In another aspect, the invention features a method of identifying a compound for treating inflammatory diseases. The method includes contacting a compound with a system (a cell system or a cell-free system) containing an HM74 and/or HM74A gene or an HM74 and/or HM74A gene product, and determining the level of HM74 and/or HM74A gene expression or protein activity in the system. The level of HM74 and/or HM74A gene expression or protein activity in the presence of the compound, if lower than that in the absence of the compound, indicates that the compound is a candidate for treating inflammatory diseases. Such a compound can be any molecule, such as a polynucleotide, RNA intereference agent, an anti-sense RNA, siRNA, an antibody or its variant, or a protein, peptide, peptidomimetic, peptoid, or a non-peptidyl molecule.

Also within the scope of the invention is a method of treating inflammatory diseases. The method includes administering to the subject an amount of a compound effective to decrease the level of HM74 and/or HM74A gene expression or protein activity in the subject. Such a compound can be any molecule, such as a polynucleotide, RNA interference agent, an anti-sense RNA, siRNA, an antibody or its variant, or a protein, peptide, peptidomimetic, peptoid, or a non-peptidyl molecule. This method optionally includes a previous step of identifying a subject suffering from or being at risk for developing inflammatory diseases by using the procedures described herein. The inflammatory diseases that may be treated include psoriasis, Crohn's disease, ulcerative colitis, multiple sclerosis or irritable bowel disease.

The invention further features a pharmaceutical composition containing a pharmaceutically acceptable carrier and an effective amount of a compound that decreases the level of HM74 and /or HM74A gene expression in a subject. The compound, when administered to a subject in need thereof, decreases the level of HM74 and/or HM74A gene expression or protein activity in the subject. Thus, the pharmaceutical composition of the invention can be used for treating inflammatory diseases.

In another aspect, the invention features a packaged product including a container, an effective amount of the compound that decreases the level of HM74 and/or HM74A gene expression or protein activity in a subject, and a legend associated with the container and indicating administration of the molecule for treating inflammatory diseases. The product can be administrated oral delivery, intravenous infusion or subcutaneous administration at different dosages.

The details of one or more embodiments of the invention are set forth in the accompanying description below. Other features, objects, and advantages of the invention will be apparent from the detailed description, and from the claims.

### FIGURES

FIG. 1: mRNA expression of HM74 and HM74A in colon tissues from normal or IBD (ulcerative colitis_patients) by TaqMan real time RT-PCR analysis.

FIG. 2: HM74/HM74A mRNA expression is induced in inflamed skin tissues from human Psoriasis patients.

FIG. 3: HM74/HM74A mRNA expression is induced in peripheral blood mononuclear cells (PBMC) from human multiple sclerosis patients.

FIG. 4: HM74/HM74A is induced by different stimuli in human primary monocytes and neutrophils.

FIG. 5: HM74 and HM74A mRNA Expression in Human Th1 and Th2 cells (TaqMan)

### DETAILED DESCRIPTION

This invention is based on the discovery that some GPCR genes, HM74 and HM74A are up-regulated in inflammatory disease tissue cells. Accordingly, the invention provides methods for diagnosing and treating inflammatory diseases by targeting these GPCR genes.

A diagnostic method of the invention involves comparing the gene expression or protein activity level of HM74 and/or HM74A in a sample prepared from a subject with that in a sample prepared from a normal subject, i.e., a subject who does not suffer from inflammatory diseases. A higher gene expression or protein activity level of HM74 and/or HM74A indicates that the subject is suffering from or at risk for developing inflammatory diseases. For example, if the gene expression level in a test subject is significant different than that in a normal subject as determined by the method described in the examples below or any analogous methods, the test subject is identified as being suffering from or at risk for developing inflammatory diseases. The method of the invention can be used on its own or in conjunction with other procedures to diagnose inflammatory diseases.

The gene expression level of HM74 and/or HM74A can be determined at either the mRNA level or the protein level. Methods of measuring mRNA levels in a tissue sample are known in the art. In order to measure mRNA levels, cells can be lysed and the levels of mRNA in the lysates or in RNA purified or semi-purified from the lysates can be determined by any of a variety of methods including, without limitation, hybridization assays using detectably labeled gene-specific DNA or RNA probes and quantitative or semi-quantitative RT-PCR or TaqMan real time PCR methodologies using appropriate gene-specific oligonucleotide primers. Alternatively, quantitative or semi-quantitative in situ hybridization assays can be carried out using, for example, tissue sections or unlysed cell suspensions, and detectably (e.g., fluorescently or enzyme) labeled DNA or RNA probes. Additional methods for quantifying mRNA include RNA protection assay (RPA) and SAGE.

Methods of measuring protein levels in a tissue sample are also known in the art. Many such methods employ antibodies (e.g., monoclonal or polyclonal antibodies) that bind specifically to the target protein. In such assays, the antibody itself or a secondary antibody that binds to it can be detectably labeled. Alternatively, the antibody can be conjugated with biotin, and detectably labeled avidin (a polypeptide that binds to biotin) can be used to detect the presence of the biotinylated antibody. Combinations of these approaches (including “multi-layer sandwich” assays) familiar to those in the art can be used to enhance the sensitivity of the methodologies. Some of these protein-measuring assays (e.g., ELISA or Western blot) can be applied to lysates of cells, and others (e.g., immunohistological methods or fluorescence flow cytometry) applied to histological sections or unlysed cell suspensions. Methods of measuring the amount of label depend on the nature of the label and are well known in the art. Appropriate labels include, without limitation, radionuclides (e.g., .sup.125I, .sup.131I, .sup.35S, .sup.3H, or .sup.32P), enzymes (e.g., alkaline phosphatase, horseradish peroxidase, luciferase, or .beta.-glactosidase), fluorescent moieties or proteins (e.g., fluorescein, rhodamine, phycoerythrin, GFP, or BFP), or luminescent moieties (e.g., Qdot.™. nanoparticles supplied by the Quantum Dot Corporation, Palo Alto, Calif.). Other applicable assays include quantitative immunoprecipitation or complement fixation assays.

The protein activity level of HM74 and/or HM74A can be determined, e.g., by measuring the binding of nicotinic acid, or by measuring GDP-GTP exchange on G-protein subunits following ligand-induced activation of HM74/HM74A See, Peltonen et al. (1998) Eur J Pharmacol 355, 275; Tunaru et al (2003) Nature Medicine 9, 352-355; Wise et al, (2003) J Biol Chem 278, 9869-9874.

The invention also provides a method for identifying and manufacturing molecules (such as, polynucleotides, RNA interference agent, an anti-sense RNA, siRNA, proteins, peptides, peptidomimetics, peptoids, antibodies or their variants, or non-peptidyl small molecules) that decrease the gene expression or protein activity level of HM74 and/or HM74A in a system. Molecules thus identified can be used, e.g., for treating inflammatory diseases.

The candidate molecules can be obtained using any of the numerous approaches in combinatorial library methods known in the art. Such libraries include: peptide libraries, peptoid libraries (libraries of molecules having the functionalities of peptides, but with a novel, non-peptide backbone that is resistant to enzymatic degradation); spatially addressable parallel solid phase or solution phase libraries; synthetic libraries obtained by deconvolution or affinity chromatography selection; and the “one-bead one-compound” libraries. See, e.g., Zuckermann et al. (1994) J Med Chem 37, 2678-2685; and Lam (1997) Antiinflammatory diseases Drug Des 12, 145.

Examples of methods for the synthesis of molecular libraries can be found in the art, for example, in: DeWitt et al. (1993) PNAS USA 90, 6909; Erb et al. (1994) PNAS USA 91, 11422; Zuckermann et al. (1994) J Med Chem 37, 2678; Cho et al. (1993) Science 261, 1303; Carrell et al. (1994) Angew Chem Int Ed Engl 33, 2059; Carell et al. (1994) Angew Chem Int Ed Engl 33, 2061; and Gallop et al. (1994) J Med Chem 37, 1233. Methods of making monoclonal and polyclonal antibodies and fragments thereof are also known in the art. See, for example, Harlow and Lane, (1988) Antibodies: A Laboratory Manual, Cold Spring Harbor Laboratory, New York. The term “antibody” includes intact molecules and fragments thereof, such as Fab, F(ab′).sub.2, and Fv which are capable of binding to an epitopic determinant present in the HM74 and/or HM74A protein.

Libraries of molecules may be presented in solution (e.g., Houghten (1992) Biotechniques 13, 412-421), or on beads (Lam (1991) Nature 354, 82-84), chips (Fodor (1993) Nature 364, 555-556), bacteria (U.S. Pat. No. 5,223,409), spores (U.S. Pat. No. 5,223,409), plasmids (Cull et al. (1992) PNAS USA 89, 1865-1869), or phages (Scott and Smith (1990) Science 249, 386-390; Devlin (1990) Science 249, 404-406; Cwirla et al. (1990) PNAS USA 87, 6378-6382; Felici (1991) J Mol Biol 222, 301-310; and U.S. Pat. No. 5,223,409).

To identify molecules that decrease the gene expression or protein activity level of HM74 and/or HM74A in a subject, a system containing the HM74 and/or HM74A gene or an HM74 and/or HM74A gene product (mRNA or protein) is contacted with a candidate compound, and the gene expression or protein activity level of HM74 and/or HM74A is evaluated relative to that in the absence of the candidate compound. In a cell system, the cell (e.g., a inflammatory diseases cell) can be a cell that naturally expresses the HM74 and/or HM74A gene, or a cell that is modified to express a recombinant HM74 and/or HM74A gene, for example, by having the HM74 and/or HM74A gene fused to a heterologous promoter or by having the HM74 and/or HM74A promoter fused to a heterologous gene. The gene expression or protein activity level of HM74 and/or HM74A can be determined according to the methods described in the examples below, or any other methods well known in the art. If the gene expression or protein activity level of HM74 and/or HM74A is lower in the presence of the candidate molecule than that in the absence of the candidate compound, the candidate molecule is identified as being useful for treating inflammatory diseases.

This invention further provides a method for treating inflammatory diseases. Subjects to be treated can optionally be identified, for example, by determining the gene expression or protein activity level of HM74 and/or HM74A in a sample prepared from a subject by methods described above. If the gene expression or protein activity level of HM74 and/or HM74A is higher in the sample from the subject than that in a sample from a normal subject, the subject is a candidate for treatment with an effective amount of a compound that decreases the gene expression or protein activity level of HM74 and/or HM74A in the subject. This method can be performed alone or in conjunction with other drugs or therapy.

The term “treating” is defined as administration of a composition to a subject, who has inflammatory diseases, with the purpose to cure, alleviate, relieve, remedy, prevent, or ameliorate the disorder, the symptom of the disorder, the disease state secondary to the disorder, or the predisposition toward the disorder. An “effective amount” is an amount of the composition that is capable of producing a medically desirable result, e.g., as described above, in a treated subject.

In one in vivo approach, a therapeutic composition (e.g., a composition containing a compound identified as described above) is administered to the subject. Generally, the molecule is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily. For treatment of inflammatory diseases, the compound can be delivered directly to the inflammatory diseases tissue.

The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100 mg/kg. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery. If the compound is poorly soluble, known surfactant materials may be used to enhance solubility of the compound. See, for example, Li et al., U.S. Application Publication No. 2006/0068007 A1, incorporated herein by reference and describing a modified Vitamin E TPGS as a surfactant material.

Alternatively, a polynucleotide, such as one containing a nucleic acid sequence encoding an anti-sense HM74 and/or HM74A RNA, can be delivered to the subject, for example, by the use of polymeric, biodegradable microparticle or microcapsule delivery devices known in the art. Another way to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells (Cristiano et al. (1995) J Mol Med 73, 479). Alternatively, tissue specific targeting can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

The above-described polynucleotide can be an RNA interference agent, i.e., a duplex-containing RNA or a DNA sequence encoding it, which inhibits the expression of HM74 and/or HM74A via RNA interference. RNA interference (RNAi) is a process in which double-stranded RNA (dsRNA) directs homologous sequence-specific degradation of messenger RNA. In mammalian cells, RNAi can be triggered by 19 to 21-nucleotide duplexes of small interfering RNA (siRNA) without activating the host interferon response. As RNAi represses the expression of a specific gene, it can be used to treat a disease caused by abnormally high levels of expression of the gene. A duplex-containing RNA can be synthesized by techniques well known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. It can also be transcribed from an expression vector and isolated using standard techniques.

In the above-mentioned polynucleotides (e.g., expression vectors), the nucleic acid sequence encoding an RNAi agent or an anti-sense HM74 and/or HM74A RNA is operatively linked to a promoter or enhancer-promoter combination. Enhancers provide expression specificity in terms of time, location, and level. Unlike a promoter, an enhancer can function when located at variable distances from the transcription initiation site, provided a promoter is present. An enhancer can also be located downstream of the transcription initiation site.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. As is well known in the medical art, the dosage for any one subject depends upon many factors, including the subject's weight, body surface area, age, the particular compound to be administered, sex, time and route of administration, general health, and other drugs being administered concurrently. Dosages will vary, but a preferred dosage for administration of polynucleotide is about 10⁶ to 10¹² copies of the polynucleotide molecule. This dose can be repeatedly administered as needed. Routes of administration can be any of those listed above.

Also within the scope of the invention is a pharmaceutical composition that contains a pharmaceutically acceptable carrier and an effective amount of a compound that decreases the gene expression or protein activity level of HM74 and/or HM74A in a subject. The pharmaceutical composition can be used to treat inflammatory diseases. The pharmaceutically acceptable carrier includes a solvent, a dispersion medium, a coating, an antibacterial and antifungal agent, and an isotonic and absorption delaying agent. The molecule can also be packaged in a container with a label or an insert to indicate the intended uses of the compound, i.e., treatment of inflammatory diseases.

The molecule of the invention can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the ligand with a solid carrier and a lubricant. Examples of solid carriers include starch and sugar bentonite.

The molecule can also be administered in a form of a hard shell tablet or a capsule containing a binder, e.g., lactose or mannitol, a conventional filler, and a tableting agent. The pharmaceutical composition can be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic agent.

The efficacy of a composition of the invention can be evaluated both in vitro and in vivo. For example, the composition can be tested for its ability to decrease the level of HM74 and/or HM74A gene expression or protein activity in vitro. For in vivo studies, the composition can be injected into an animal (e.g., an animal model) and its effects on inflammatory diseases are then accessed. Based on the results, an appropriate dosage range and administration route can be determined.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

### Experimental Section:

### EXAMPLE 1

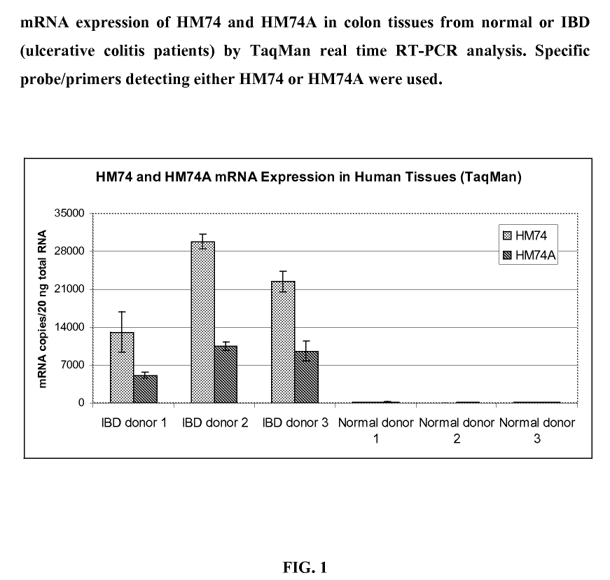

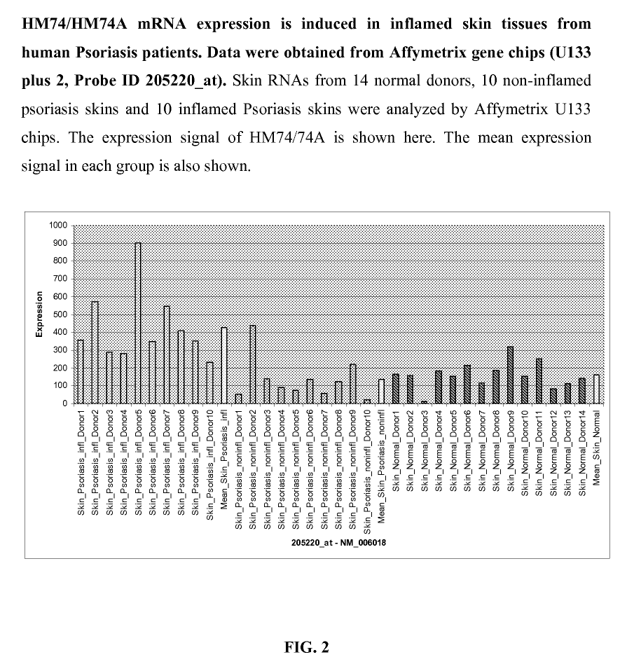

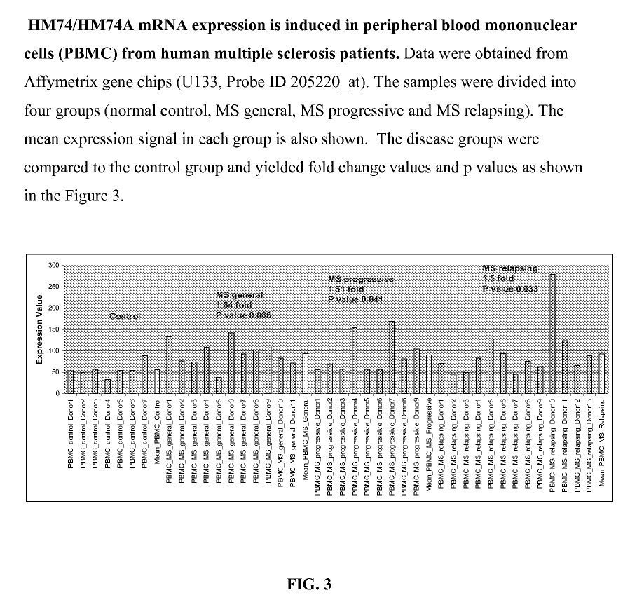

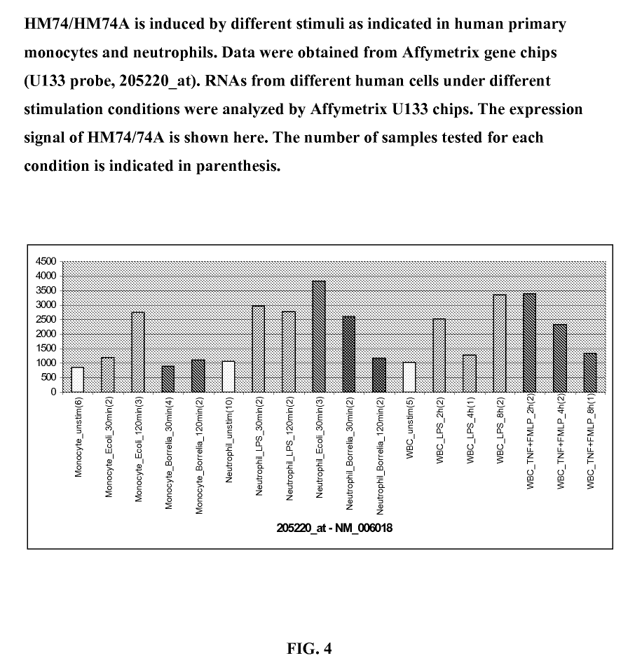

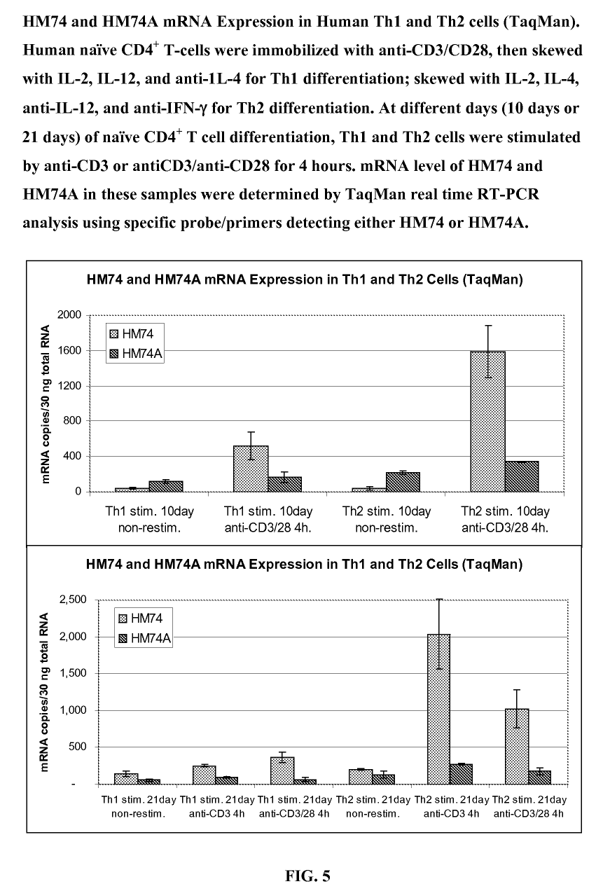

We applied Affymetrix gene chips technologies (human U133 chips) to perform gene expression profile studies of the human colon tissues from ulcerative colitis patients and normal donors. We observed that HM74 is highly induced in inflamed colon tissues from ulcerative colitis patients. Further statistical analysis of colon tissues from inflamed bowel disease (IBD) patients and normal donors (9 Crohn's disease patients, 17 Ulcerative colitis patients, 211 normals) revealed that HM74 is induced 4.4 fold (p value, 0.002) in Crohn's disease colons and induced 2.5 fold (p value, 0.006) in Ulcerative colitis colons. The oligonucleotide probes (probe ID 205220_at) on Affymetrix chips detect both the human HM74 and HM74A. In order to identify which HM74 isoform is induced in IBD, we performed TaqMan real time RT-PCR analysis with specific TaqMan primers and probes designed for HM74 or HM74A. As shown in FIG. 1, both HM74 and HM74A are expressed in inflamed colon tissues from ulcerative colitis patients, but not from normal colons. In addition, gene chip data (Probe ID 205220_at) show that the expression of HM74/74A are highly induced in inflamed skin tissues from Psoriasis patients than normal skin tissues or non-lesion Psoriasis skin tissues (FIG. 2). Moreover, HM74/HM74A mRNA expression is also induced in peripheral blood mononuclear cells (PBMC) from human multiple sclerosis patients compared to PBMC from normal donors (FIG. 3). HM74/HM74A is also highly induced by bacteria and LPS in human primary monocytes and neutrophils (FIG. 4), suggesting their function in mediating inflammatory responses in monocytes and neutrophils. We performed TaqMan real time RT-PCR analysis in human Th1 and Th2 CD4⁻ T cells with specific TaqMan primers and probes designed for HM74 or HM74A. As shown in FIG. 5, HM74, but not HM74A, is significantly induced in human Th1 and Th2 cells by anti-CD3 or anti-CD3/anti-CD28 stimulation. The stimulation of HM74 mRNA is higher in Th2 cells than in Th1 cells (FIG. 5). The dissociation of HM74 and HM74A expression has never been reported previously. Taken together, HM74 and HM74A are highly induced in inflammatory cells and in inflammatory diseases, and would serve as attractive targets for inflammatory disease including inflammatory bowel disease, psoriasis and multiple sclerosis. Since HM74/HM74A is a G-protein coupled receptor, small molecule agonists or antagonists may have anti-inflammatory and immunosuppression effects.

Total RNA isolation: Total cellular RNA was isolated from tissue or cell samples using the RNeasy Kits and RNase-Free DNase Set Protocol according to manufacturer's description (QIAGEN).

TaqMan probes and primers: PCR primers and TaqMan probes were designed using Primer Express 1.5 Software (Applied Biosystems). The TaqMan probes were labeled with a reporter fluorescent dye, FAM (6-carboxyfluorescein), at the 5′ end and a fluorescent dye quencher TAMRA (6-carboxy-tetramethyl-rhodamine) at the 3′ end. The specificity of PCR primers was tested under normal PCR conditions in a thermal cycler prior to TaqMan PCR quantitation.

Reverse transcription: RT reactions were carried out for each RNA sample in MicroAmp reaction tubes using TaqMan reverse transcription reagents. Each reaction tube contained 500 ng of total RNA in a volume of 50 μl containing 1× TaqMan RT buffer, 5.5 mM MgCl₂, 500 μM of each dNTP, 2.5 μM of Random Hexamers or oligo-d(T)₁₆ primers, 0.4 U/μl of RNase inhibitor, and 1.25 U/μl of MultiScribe Reverse Transcriptase. RT reactions were carried out at 25° C. for 10 min, 48° C. for 40 min and 95° C. for 5 min [Note: the incubation at 25° C. for 10 min is necessary for the RT reaction with random hexamers or oligo-d(T)₁₆ primers to obtain the optimal results]. The RT reaction mixture was then placed at 4° C. for immediate use of PCR amplification, or stored at −20° C. for later use (similar results are expected at these two different temperatures of storage).

Construction of standard curve: To determine the copy numbers of the target transcript, a human genomic DNA (Clontech) was used to generate a standard curve. The copy numbers of genomic DNA template were calculated according to the molecular weight of human diploid genome [3×10⁹ bp=3×10⁹×660 (M.W.)=2×10¹² g], and then 1 μg/μl genomic DNA was converted into 2.4×10⁶ copy numbers based upon the Avogadro's number (1 mol=6.022×10²³ molecules). The genomic DNA was serially (every ten-fold) diluted at a range of 5×10⁵ to 5×10⁰ copy numbers. Each sample was run in triplicates, and the Rₙ (the ratio of the amount of reporter dye emission to the quenching dye emission) and threshold cycle (Cₜ) values were averaged from each reaction.

TaqMan real-time quantitative PCR: The principle of the TaqMan real-time detection is based on the fluorogenic 5′ nuclease assay. A thermal stable AmpliTaq Gold DNA polymerase was used for the PCR amplification. Real-time RT-PCR was performed in a MicroAmp Optical 96-Well Reaction Plate (Applied Biosystems). Each well contained 2 μl of each RT product (20 ng total RNA), 1× TaqMan buffer A, 5.5 mM MgCl₂, 200 μM dATP/dCTP/dGTP, 400 μM dUTP, 200 nM primers (forward and reverse), 100 nM TaqMan probe, 0.01 U/μl AmpErase, and 0.025 U/μl AmpliTaq Gold DNA polymerase in a total volume of 25 μl. Each well was closed with MicroAmp Optical caps (Applied Biosystems), following complete loading of reagents. Amplification conditions were 2 min at 50° C. (for AmpErase UNG incubation to remove any uracil incorporated into the cDNA), 10 min at 95° C. (for AmpliTaq Gold activation), and then run for 40 cycles at 95° C. for 15 s, 60° C. for 1 min. All reactions were performed in the ABI Prism 7700 Sequence Detection System for the test samples, standards, and no template controls. They were run in triplicates using the Sequence Detector V 1.6 program. The Rₙ and Cₜ were averaged from the values obtained in each reaction. A “standard curve” was constructed by plotting the Cₜ vs. the known copy numbers of the template in the standard. According to the standard curve, the copy numbers for all unknown samples were obtained automatically.

Normalization of mRNA expression level: The copy numbers of mRNA in each sample were calculated based on its Cₜ value with its plasmid DNA standard curve. The copy numbers were then normalized to Gapdh to minimize variability in the results due to differences in the RT efficiency and RNA integrity among test samples.

Primer sequences used for Quantitative RT-PCR

HM74 Primers
HM74_F

| | |
|---|---|
| Sequence: | 5′-ACTACTATGTGCGGCGTTCAGAC-3′ |
| HM74_R | |
| Sequence: | 5′-GGCGGTTCATGGCAAACA-3′ |

HM74 TaqMan ® probe:
HM74_T

| | |
|---|---|
| Sequence: | 5′FAM-ACCAGCCGGCAAGGGATGTCC-TAMRA3′ |

HM74A primers
HM74A_F

| | |
|---|---|
| Sequence: | 5′-ACAACTATGTGAGGCGTTGGGA-3′ |
| HM74A_R | |
| Sequence: | 5′-TGGCGGTTCATAGCCAACA-3′ |

HM74A TaqMan ® probe:
HM74A_T

| | |
|---|---|
| Sequence: | 5′FAM-ATCAGCCGGCAAGGGATGTCC-TAMRA3′ |

### Results

We observed that HM74 is highly induced in inflamed colon tissues from ulcerative colitis patients. Further statistical analysis of colon tissues from inflamed bowel disease (IBD) patients and normal donors (9 Crohn's disease patients, 17 Ulcerative colitis patients, 211 normals) revealed that HM74 is induced 4.4 fold (p value, 0.002) in Crohn's disease colons and induced 2.5 fold (p value, 0.006) in Ulcerative colitis colons. As shown in FIG. 1, both HM74 and HM74A are expressed in inflamed colon tissues from ulcerative colitis patients, but not from normal colons. As shown in FIG. 5, HM74, but not HM74A, is significantly induced in human Th1 and Th2 cells by anti-CD3 or anti-CD3/anti-CD28 stimulation.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

We claim:

1. A method of determining whether a test subject is suffering from or at risk for developing an inflammatory disease comprising:
(1) providing a tissue sample from a test subject and determining the gene expression level or protein activity level of HM74 and/or HM74A in the sample;
(2) comparing the gene expression level or protein activity level of HM74 and/or HM74A in said sample to the gene expression level or protein activity level of HM74 and/or HM74A in a tissue sample from a normal subject; and
(3) if the gene expression level or protein activity level of HM74 and/or HM74A in the sample from the test subject is higher than that in the sample from the normal subject, it indicates that the test subject is suffering from or at risk for developing an inflammatory disease.

2. A method according to claim 1, wherein the inflammatory disease is psoriasis, Crohn's disease, ulcerative colitis, multiple sclerosis or irritable bowel disease.

3. A method according to claim 1, wherein the gene expression level of HM74 and/or HM74A is determined by measuring the amount of HM74 mRNA and/or HM74A mRNA in the sample.

4. A method according to claim 1, wherein the gene expression level of HM74 and/or HM74A is determined by measuring the amount of HM74 protein and/or HM74A protein in the sample.

5. A method according to claim 4, wherein the protein is that defined by Seq. ID NO. 3 and/or Seq. ID NO. 4.

6. A method according to claim 1, wherein the protein activity level of HM74 and/or HM74A is determined by measuring the binding of nicotinic acid.

7. A method according to claim 1, wherein the protein activity level of HM74 and/or HM74A is determined by measuring GDP-GTP exchange on G-protein subunits following ligand-induced activation of HM74/HM74A.

8. A method according to claim 1, further comprising treating an inflammatory disease in said subject comprising administering to said subject an amount of a compound effective to decrease the level of HM74 and/or HM74A gene expression or protein activity in the subject.

9. A method according to claim 8, wherein said compound is a polynucleotide, RNA interference agent, an anti-sense RNA, siRNA, an antibody or its variant, a protein, peptide, peptidomimetic, peptoid, or a non-peptidyl molecule.

10. A method of identifying a compound for treating inflammatory diseases, said method comprising:
(1) contacting a compound with a system containing an HM74 and/or HM74A gene or an HM74 and/or HM74A gene product and determining the level of HM74 and/or HM74A gene expression or protein activity in the system in the presence of said compound;
(2) comparing the level of HM74 and/or HM74A gene expression or protein activity obtained in step (1) with the level of HM74 and/or HM74A gene expression or protein activity in said system in the absence of said compound; and
(3) if the level of HM74 and/or HM74A gene expression or protein activity in said system in the presence of said compound is lower than that in the absence of said compound, it indicates that the compound is useful for treating inflammatory diseases.

11. A method according to claim 10, wherein said compound is a polynucleotide, RNA interference agent, an anti-sense RNA, siRNA, an antibody or its variant, a protein, peptide, peptidomimetic, peptoid, or a non-peptidyl molecule.

12. A method according to claim 10, wherein the gene expression level of HM74 and/or HM74A is determined by measuring the amount of HM74 mRNA and/or HM74A mRNA in the sample.

13. A method according to claim 10, wherein the gene expression level of HM74 and/or HM74A is determined by measuring the amount of HM74 protein and/or HM74A protein in the sample.

14. A method according to claim 13, wherein the protein is that defined by Seq. ID NO. 3 and/or Seq. ID NO. 4.

15. A method according to claim 10, wherein the protein activity level of HM74 and/or HM74A is determined by measuring the binding of nicotinic acid.

16. A method according to claim 10, wherein the protein activity level of HM74 and/or HM74A is determined by measuring GDP-GTP exchange on G-protein subunits following ligand-induced activation of HM74/HM74A.

17. A method for treating an inflammatory disease in a subject comprising administering to a subject an amount of a compound effective to decrease the level of HM74 and/or HM74A gene expression or protein activity in the subject.

18. A method according claim 17, wherein said compound is a polynucleotide, RNA interference agent, an anti-sense RNA, siRNA, an antibody or its variant, a protein, peptide, peptidomimetic, peptoid, or a non-peptidyl molecule.

19. A method according to claim 17, wherein the inflammatory disease is psoriasis, Crohn's disease, ulcerative colitis, multiple sclerosis or irritable bowel disease.

20. A pharmaceutical composition containing a pharmaceutically acceptable carrier and an effective amount of a compound that decreases the level of HM74 and /or HM74A gene expression or protein activity in a subject.

21. A pharmaceutical composition according to claim 20, wherein said compound is a polynucleotide, RNA interference agent, an anti-sense RNA, siRNA, an antibody or its variant, a protein, peptide, peptidomimetic, peptoid, or a non-peptidyl molecule.

22. A packaged product comprising a container, a pharmaceutical composition according to claim 20, and a legend associated with the container and indicating administration of the composition for treating inflammatory diseases.
